(19) United States
(12) Patent Application Publication
(10) Pub. No.: US 2015/0224074 A1
(43) Pub. Date: Aug. 13, 2015

(54) STABLE FORMULATIONS OF FATTY ACIDS

(71) Applicant: Eric Kuhrts, Bodega, CA (US)

(72) Inventor: Eric Kuhrts, Bodega, CA (US)

(21) Appl. No.: 13/699,946

(22) PCT Filed: May 23, 2011
(86) PCT No.: PCT/US11/37611

§ 371 Date: May 14, 2013

Related U.S. Application Data
(60) Provisional application No. 61/348,119, filed on May 25, 2010.

(51) Int. Cl. A61K 31/202; A23L 2/52; A23L 1/30; A61K 47/14; A61K 47/22

(52) U.S. Cl. CPC A61K 31/202; A61K 47/14; A61K 47/22; A23L 1/3008; A23L 2/52; A23V 2002/00

(57) ABSTRACT

Methods and formulations for increasing the water solubility and/or stability of dietary fatty acids are disclosed.

### BACKGROUND

Dietary or nutritional fatty acids are a family of unsaturated fatty acids that include the omega-3 fatty acids, such as eicosapentaenoic acid (EPA) and docosahexaenoic acid (DHA), as well as omega-6 and omega-9 fatty acids. One source for omega-3 fatty acids is fish oil; however, omega-3 fatty acids can also be obtained from botanical sources and algae. With respect to omega-3 fatty acids, many cardiovascular and other health benefits are known, in addition to their significance in nutrition. In fact, consumption of nutritional or dietary fatty acids have been identified with many health benefits, having the potential to impact numerous diseases such as cardiovascular, neurological, immune function, and arthritis. Due to the increased awareness of the health benefits of the omega-3 class of fatty acids, dietary food supplements of fish oil and flax oil have become popular. With the availability of deodorized fish oils, it is now possible to make beverages containing omega-3 fatty acids, or fish oil, but the stability of the oils remains a problem. As such, it would be advantageous to provide a more stable, water-soluble formulation of these fatty acids for use in beverages. Such a product would have better shelf-life characteristics and more desirable sensory qualities for consumers.

### SUMMARY

In one aspect, the present disclosure provides a water-soluble formulation, comprising a dietary fatty acid; a non-ionic surfactant; a phenolic amine; and optionally, water.

In another embodiment, a method of stabilizing dietary fatty acids in water can comprise warming a non-ionic surfactant; combining a dietary fatty acid with the non-ionic surfactant to form a surfactant-dietary fatty acid mixture; and combining the surfactant-dietary fatty acid mixture with water containing a phenolic amine to form a stabilized, clear, water-soluble, self-assembled fatty acid solution. In one example, the step of warming can be at a temperature from about 80° F. to about 200° F. In another example, the step of combining the surfactant-dietary fatty acid mixture with water/phenolic amine can be at a rate from about 0.05 mL/sec to about 25.0 mL/sec.

In another embodiment, a method for enhancing the stability of a dietary fatty acid can comprise combining a dietary fatty acid and a non-ionic surfactant to form a surfactant-dietary fatty acid mixture; and combining the surfactant-dietary fatty acid mixture with a water soluble phenolic amine dissolved in water. This can result in a composition having enhanced stability of the dietary fatty acid in water compared to the stability of the dietary fatty acid in water alone.

In another embodiment, a method of making a stable, water-soluble pharmaceutical gel composition of a dietary fatty acid can comprise heating a water-soluble non-ionic surfactant in a container to a temperature of about 80° F. to about 200° F. while mixing the non-ionic surfactant until a clear non-ionic surfactant is formed; and adding a dietary fatty acid to the clear non-ionic surfactant to form a dietary fatty acid and non-ionic surfactant combination. An additional step includes stirring the dietary fatty acid and non-ionic surfactant combination until thoroughly mixed so as to constitute from 0.1 wt % to 25wt % dietary fatty acid and from 70 wt % to 99.9 wt % non-ionic surfactant, wherein the dietary fatty acid is sufficiently dispersed or dissolved in the non-ionic surfactant so that a gel composition containing no visible micelles or particles of dietary fatty acid is formed. Additional steps include dissolving a pyridoxamine salt in water in a second vessel and adding the gel composition to warm water containing the dissolved pyridoxamine salt at a rate not to exceed 5% of the volume of water per second while continuously stirring the water until a clear solution is formed.

In another example, a method of enhancing the stability of a dietary fatty acid in a subject can comprise combining a dietary fatty acid and a non-ionic surfactant with a phenolic amine and water to form a mixture of non-ionic surfactant, dietary fatty acid, phenolic amine, and water; and administering the mixture to a subject, thereby enhancing the stability of the dietary fatty acid or dietary fatty acid metabolite.

In each of the embodiments described herein there are many variables that can be applicable to the formulation and related methods. For example, the dietary fatty acid can include a single dietary fatty acid, or alternatively, it includes multiple dietary fatty acids. The dietary fatty acid may also be, for example, an omega-3 fatty acid, such as eicosapentaenoic acid (EPA), docosahexaenoic acid (DHA), or combination thereof. The formulation can be non-alcoholic and/or non-aprotic. Exemplary concentrations of dietary fatty acid(s) can be from at least about 0.01 mg/mL, at least about 1 mg/mL, at least about 0.01% by weight, or at least about 25% by weight of the formulation. The formulation can likewise comprise from about 1 mg to about 250 mg of dietary fatty acid, or in an alternative embodiment, at least about 10 mg of dietary fatty acid.

The non-ionic surfactant can be non-ionic water soluble mono-, di-, and tri-glycerides; ethoxylated castor oil; non-ionic water soluble mono- and di- fatty acid esters of polyethylene glycol; non-ionic water soluble sorbitan fatty acid esters; polyglycolyzed glycerides; non-ionic water soluble triblock copolymers; and/or derivates thereof. More specific examples include glycerol-polyethylene glycol oxystearate; macrogolglycerol ricinoleate; macrogolglycerol hydroxystearate; and/or polyethylene glycol 660 hydroxystearate. Furthermore, the phenolic amine can be a pyridoxamine, a pyridoxamine analogue, or a salt thereof.

The formulations can be oral formulation, such as in the form of a soft gel capsule or a beverage. The formulation can also be in the form of a spray or a topical. Dietary fatty acids for use in accordance with embodiments of the present disclosure can be derived from fish, algae, or vegetable sources.

In accordance with some examples, formulations can be prepared that do not exhibit a peroxide value exceeding about 2.0 meq/Kg during one week of storage under refrigerated conditions, and in some embodiments, it is less than 1.0 meq/Kg. Likewise, the formulation does not exhibit a peroxide value exceeding about 5.0 meq/Kg during 30 days of sealed storage under ambient conditions. In other words, the formulations are very stable compared to these oils when not prepared as described herein.

In other embodiments, it is noted that the formulation can be prepared as a concentrate, or as a ready to use formulation.

### DETAILED DESCRIPTION

Reference will now be made to the examples illustrated, and specific language will be used herein to describe the same. It will nevertheless be understood that no limitation of the scope of the technology is thereby intended. Additional features and advantages of the technology will be apparent from the detailed description which follows, which illustrate, by way of example, features of the technology. The abbreviations used herein have their conventional meaning within the chemical and biological arts.

The “salts” described herein can be pharmaceutically acceptable salts in some examples. “Pharmaceutically acceptable salts” include salts of the active compounds which are prepared with nontoxic acids or bases, depending on the particular substituent moieties found on the compounds described herein. When formulations of the present disclosure contain acidic functionalities, base addition salts can be obtained by contacting the neutral form of such compounds with a sufficient amount of the desired base, either neat or in a suitable inert solvent. Examples of pharmaceutically acceptable base addition salts include sodium salts, potassium salts, calcium salts, ammonium salts, organic amino salts, magnesium salts, and the like. When formulations of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids including, but not limited to, hydrochloric acid, hydrobromic acid, nitric acid, carbonic acid, monohydrogencarbonic acid, phosphoric acid, monohydrogenphosphoric acid, dihydrogenphosphoric acid, sulfuric acid, monohydrogensulfuric acid, hydriodic acid, phosphorous acids and the like, as well as the salts derived from relatively nontoxic organic acids including, but not limited to, acetic acid, propionic acid, isobutyric acid, maleic acid, malonic acid, benzoic acid, succinic acid, suberic acid, fumaric acid, lactic acid, mandelic acid, phthalic acid, benzenesulfonic acid, p-tolylsulfonic acid, citric acid, tartaric acid, methanesulfonic acid, and the like. Also included are salts of amino acids such as arginate and the like, and salts of organic acids such as glucuronic or galactunoric acids and the like (see, for example, Berge et al., “Pharmaceutical Salts”, Journal of Pharmaceutical Science, 1977, 66, 1-19). Certain specific formulations of the present disclosure contain both basic and acidic functionalities that allow the compounds to be converted into either base or acid addition salts. The neutral forms of the compounds can be regenerated by contacting the salt with a base or acid and isolating the parent compound in the conventional manner. The parent form of the compound differs from the various salt forms in certain physical properties, such as solubility in polar solvents.

“Dietary fatty acids” as used herein includes nutritional fatty acids, omega-3 fatty acids derived from natural sources such as fish, algae or vegetable sources, including botanical sources such as chia, sage, Salvia hispanica, or flax sources derived from linseed, or produced synthetically. The following is a list of omega-3 fatty acids (Table 1) followed by a list of botanical extracts of omega-3 fatty acids (Table 2). These lists are exemplary only, and are not considered to be limiting.

TABLE 1
List of several common n-3 fatty acids found in nature

| Common Name | Lipid Name | Chemical Name |
|---|---|---|
| — | 16:3 (n-3) | all-cis-7,10,13- |
| | | hexadecatrienoic acid |
| Alpha-Linolenic acid (ALA) | 18:3 (n-3) | all-cis-9,12,15- |
| | | octadecatrienoic acid |
| Stearidonic acid (STD) | 18:4 (n-3) | all-cis-6,9,12,15- |
| | | octadecatetraenoic acid |
| Eisosatrienoic acid (ETE) | 20:3 (n-3) | all-cis-11,14,17- |
| | | eicosatrienoic acid |
| Eicosatetraenoic acid (ETA) | 20:4 (n-3) | all-cis-8,11,14,17- |
| | | eicosatrienoic acid |
| Eicosapentaenoic acid (EPA) | 20:5 (n-3) | all-cis-5,8,11,14,17- |
| | | eicosapentaenoic acid |
| Docosapentaenoic acid (DPA), | 22:5 (n-3) | all-cis-7,10,13,16,19- |
| Clupanodonic acid | | docosapentaenoic acid |
| Docosahexaenoic acid (DHA) | 22:6 (n-3) | all-cis-4,7,10,13,16,19- |
| | | docosahexaenoic acid |
| Tetracosapentaenoic acid | 24:5 (n-3) | all-cis-9,12,15,18,21- |
| | | docosahexaenoic acid |
| Tetracosahexaenoic acid | 24:6 (n-3) | all-cis-6,9,12,15,18,21- |
| (Nisinic Acid) | | tetracosenoic acid |

TABLE 2
Sources of botanical extracts of omega-3 fatty acids

| Common Name | Alternative Name | Linnaean Name | % n-3 |
|---|---|---|---|
| Chia | Chia sage | Salvia hispanica | 64 |
| Kiwifruit | Chinese gooseberry | Actinidia chinensis | 62 |
| Perilla | Shiso | Perilla frutescens | 58 |
| Flax | Linseed | Linum usitatissimum | 55 |
| Lingonberry | Cowberry | Vaccinium vitis-idaea | 49 |
| Camelina | Gold-of-pleasure | Camelina sativa | 36 |
| Purslane | Portulaca | Portulaca oleracea | 35 |
| Black Raspberry | — | Rubus occidentalis | 33 |

Dietary Fatty Acids containing omega-3 fatty acids may also be derived from algae such as Crypthecodinium cohnii and Schizochytrium, which are rich sources of DHA, or brown algae (kelp) for EPA. They may also include conjugated linoleic acid (CLA), omega-6 fatty acids, and omega-9 fatty acids, such as linolenic acid, linoleic acid (18:2), and gamma linolenic acid (GLA, 18:3). Vegetarian polyunsaturated omega-3 fatty acid pre-cursors, such as stearidonic acid, are also included under the definition of “dietary fatty acids.” Stearidonic acid, for example, is a pre-cursor to eicosapentaeonoic acid (EPA) in humans.

A “non-ionic surfactant,” as used herein, is a surface-active agent that tends to be non-ionized (i.e. uncharged) in neutral solutions (e.g. neutral aqueous solutions).

The terms “patient’ and “subject” are used interchangeably, and refer to humans and other mammals.

As used herein, the term “titration” or “titrate” means the slow addition of a compound or solution to a liquid while mixing. The rate at which the compound or solution is added should not exceed a certain threshold, or the clear nature and viscosity of the solute is lost. Slow addition can be as a drizzle or drop by drop, but in no case will typically equal large volumes. Slow addition can be specified as a percent of the volume it is being added to per second or per minute, for example 5 mL per second to 100 mL water, or 5 wt % addition per second or minute of the content being added to water or water containing beverage.

As used herein, the term “clear aqueous solution” in reference to a solution containing dietary fatty acid means a water containing solution (e.g. a beverage) that is free of visible particles of undissolved dietary fatty acid. In accordance with some embodiments, the clear aqueous solution is not a more traditional dispersion or suspension, and remains clear upon sitting undisturbed for 1 hour or more. Often, very small micelles are formed that are not visible, and thus, the solution is clear.

The term “water-soluble” herein refers to the solubilization or very fine dispersion of dietary fatty acids so that they are not visible to the naked eye in solution. Often, in the formulations of the present disclosure, the fatty acids can form micelles in water with a non-ionic surfactant barrier, and the micelles can be smaller than about 100 nm in size, and often are about 15 nm to about 30 nm in size. Thus, whether the dietary fatty acids are strictly dissolved or merely so finely dispersed that the solution they form within is clear, this is still considered to be “water-soluble” in accordance with embodiments of the present disclosure.

As used herein, the term “oxidation” refers particularly to the degradation or spoiling of an oil or fat through exposure to air or oxygen, resulting in a loss of electrons or an increase in oxidation state. Oxidation can be the result of different chemical mechanisms during the processing, storage, or heating of an oil or fat. There are various types of oxidation, namely autooxidation, photosensitized oxidation, thermal oxidation, and enzymatic oxidation. One type of oxidation particularly relevant in the context of the present disclosure is thermal oxidation, because the formulations and process involved in this application involve heating, and thermal oxidation is one of the most rapid forms of oxidation. Various types of oxidation products are produced by autooxidation and thermal oxidation, such as hydroperoxides, aldehydes, and ketones. These degradation products can be measured, providing an analytical index for aging or stability studies for various oils under different conditions, providing a comprehensive spectrum of decomposition products.

As used herein, the term “peroxide value” or “PV” refers to a quantitative measure of the oxidation of oil. Peroxide value is usually given in meq/Kg of oil (milliequivalents per kilogram). One method used to determine PV is American Oil Chemists' Society Official Method (AOCS) Cd 8-53. The peroxide value is also a means of assessing the extent of rancidity reactions that have occurred during storage of a fat or oil. Peroxide value is defined as the amount of peroxide oxygen per kilogram of oil. Peroxide value is measured by determining the amount of iodine which is formed by the reaction of peroxides formed in the oil with iodide ion. A decrease in peroxide values leads to better sensory characteristics or quality of the oil, such as smell and taste

“Phenolic amine” includes compounds such as pyridoxamine or pyridoxamine analogs. Pyridoxamine, 4-(aminomethyl)-5-hydroxymethyl)-2-methylpyridin-3-ol, is a member of the vitamin B-6 family, which includes pyridoxal and pyridoxine. In one example, pyridoxamine, as used herein, is used in the form of the hydrochloride salt, or pyridoxamine dihydrocloride.

Concentrations, amounts, solubilities, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a concentration range of 0.5 to 400 should be interpreted to include not only the explicitly recited concentration limits of 0.5 and 400, but also to include individual concentrations within that range, such as 0.5, 0.7, 1.0, 5.2, 8.4, 11.6, 14.2, 100, 200, 300, and sub-ranges such as 0.5-2.5, 4.8-7.2, 6-14.9, 55, 85, 100-200, 117, 175, 200-300, 225, 250, and 300-400, etc. This interpretation should apply regardless of the breadth of the range or the characteristic being described.

In accordance with these definitions and other disclosure herein, as well as what is generally known in the related arts, it has been recognized that benefits may be realized from adding nutritional fatty acids to beverages or other dosage forms. Until recently, deodorized fish oils with virtually no fishy taste of smell have not been readily available to consumers. With the availability of deodorized fish oils, it is now possible to make beverages containing omega-3 fatty acids, or fish oil, but the stability of the oils in these dosage forms remains a serious problem. Normally, these oils are kept frozen to prevent or slow down oxidation. As soon as these oils are defrosted and processed, they begin to undergo oxidation. Oxidation is a natural process that occurs when oils are exposed to air or oxygen. The oxidation of oils can be measured quantitatively by measuring certain markers of oxidation such as the peroxide value (PV) or isoprostanes. Rancidification is the oxidation of fats, fatty acids, or edible oils, and most people are familiar with the term rancid to describe the change in smell associated with edible oils or fats such as butter after exposure to air for prolonged periods. A rancid oil or fat also has an objectionable taste. Oxidation is the loss of electrons or increase in oxidation state by a molecule, atom, or ion.

Once oxidized, the undesirable sensory characteristics become apparent, as odor and taste are directly correlated with oxidation. For example, the fishy odor and taste of fish oil is a highly undesirable property of a fish oil-containing beverage. It would be desirable to have a formulation of nutritional fatty acids that are soluble in water (e.g., for a beverage or soft capsule, or other dosage form), and/or a water-soluble omega-3 fatty acid formulation (e.g., fish oil) that would be stabilized, and virtually free of undesirable odor and taste. In addition, it would also be advantageous to have a process or method of making such formulations.

It has been discovered that non-ionic surfactants may be used to increase the solubility and/or bioavailability of dietary fatty acids. Thus, non-ionic surfactants may be used to form water-soluble formulations containing dietary fatty acids.

In one aspect, the present disclosure provides a water-soluble formulation including a dietary fatty acid; a non-ionic surfactant; a phenolic amine; and water. In some embodiments, the water-soluble formulation does not include a vegetable oil suspension or visible macro-micelles (micelles visible to the naked eye) in water. In other embodiments, the water-soluble formulation does not include an alcohol (e.g. the dietary fatty acid is not first dissolved in alcohol and then added to water), and/or is not an aprotic formulation.

Regarding the non-ionic surfactant specifically, useful non-ionic surfactants include, for example, non-ionic water soluble mono-, di-, and tri-glycerides; ethoxylated caster oil, non-ionic water soluble mono- and di-fatty acid esters of polyethyelene glycol; non-ionic water soluble sorbitan fatty acid esters (e.g. sorbitan monooleates such as SPAN 80 and TWEEN 20 (polyoxyethylene 20 sorbitan monooleate)); polyglycolyzed glycerides; non-ionic water soluble triblock copolymers (e.g. poly(ethyleneoxide)/poly-(propyleneoxide)/poly(ethylenenoxide), POLOXAMER 406 (PLURONIC F-127)); and derivatives thereof.

More specific examples of non-ionic water soluble mono-, di-, and tri-glycerides include propylene glycol dicarpylate/dicaprate (e.g. MIGLYOL 840), medium chain mono- and diglycerides (e.g. CAPMUL and IMWITOR 72), medium-chain triglycerides (e.g. caprylic and capric triglycerides such as LAVRAFAC, MIGLYOL 810 or 812, CRODAMOL GTCC-PN, and SOFTISON 378), long chain monoglycerides (e.g. glyceryl monooleates such as PECEOL, and glyceryl monolinoleates such as MAISINE), polyoxyl castor oil (e.g. macrogolglycerol ricinoleate, macrogolglycerol hydroxystearate, macrogol cetostearyl ether), polyethylene glycol 660 hydroxystearate, and derivatives thereof.

Non-ionic water soluble mono- and di-fatty acid esters of polyethyelene glycol include d-α-tocopheryl polyethyleneglycol 1000 succinate (TPGS), poyethyleneglycol 660 12-hydroxystearate (SOLUTOL HS 15), polyoxyl oleate and stearate (e.g. PEG 400 monostearate and PEG 1750 monostearate), and derivatives thereof.

Polyglycolyzed glycerides include polyoxyethylated oleic glycerides, polyoxyethylated linoleic glycerides, polyoxyethylated caprylic/capric glycerides, and derivatives thereof. Specific examples include LABRAFIL M-1944CS, LABRAFIL M-2125CS, LABRASOL, SOFTIGEN, and GELUCIRE.

In some embodiments, the non-ionic surfactant is a glycerol-polyethylene glycol oxystearate, or derivative thereof. These compounds may be synthesized by reacting either castor oil or hydrogenated castor oil with varying amounts of ethylene oxide. Macrogolglycerol ricinoleate is a mixture of 83 wt % relatively hydrophobic and 17 wt % relatively hydrophilic components. The major component of the relatively hydrophobic portion is glycerol polyethylene glycol ricinoleate, and the major components of the relatively hydrophilic portion are polyethylene glycols and glycerol ethoxylates. Macrogolglycerol hydroxystearate (glycerol-polyethylene glycol oxysterate) is a mixture of approximately 75 wt % relatively hydrophobic of which a major portion is glycerol polyethylene glycol 12-oxystearate.

In some embodiments, the water-soluble formulations include the dietary fatty acid, and glycerol-polyethylene glycol oxystearate, to form a transparent water-soluble formulation. A “transparent” water-soluble formulation or “clear aqueous solution” refers to a formulation that can be clearly seen through with the naked eye and is optionally colored. In some embodiments, the transparent water-soluble formulations do not contain particles (e.g. particles of undissolved dietary fatty acid) visible to the naked eye. Thus, in some embodiments, the transparent water-soluble formulations are not opaque, cloudy or milky-white. Transparent water-soluble formulations disclosed herein do not include milky-white emulsions or suspensions in vegetable oil such as corn oil. Transparent water-soluble formulations are also typically not formed by first dissolving the dietary fatty acid in alcohol, or other organic solvents, and then mixed with water.

As mentioned as well, in some embodiments, the water-soluble formulation is a non-alcoholic formulation. A “non-alcoholic” formulation, as used herein, is a formulation that does not include (or includes only in trace amounts) methanol, ethanol, propanol or butanol. For example, the formulation does not include (or includes only in trace amounts) ethanol or other similar alcohols.

In other embodiments, the formulation can be a non-aprotic solvated formulation. The term “non-aprotic solvated,” as used herein, means that water soluble aprotic solvents are absent or are included only in trace amounts. Water soluble aprotic solvents are water soluble non-surfactant solvents in which the hydrogen atoms are not bonded to an oxygen or nitrogen and therefore cannot donate a hydrogen bond. Thus, the water-soluble formulation in these embodiments do not include (or includes only in trace amounts) such aprotic solvents.

Polar aprotic solvents are aprotic solvents whose molecules exhibit a molecular dipole moment but whose hydrogen atoms are not bonded to an oxygen or nitrogen atom. Examples of polar aprotic solvents include aldehydes, ketones, dimethyl sulfoxide (DMSO), and dimethyl formamide (DMF). In other embodiments, the water soluble formulation does not include (or includes only in trace amounts) dimethyl sulfoxide. Thus, in some embodiments, the water soluble formulation does not include polar aprotic solvents, such as DMSO. In a related embodiment, the water soluble formulation does not include DMSO or ethanol.

The formulation can also be devoid (or include only trace amounts) of non-polar aprotic solvents. Non-polar aprotic solvents are aprotic solvents whose molecules exhibit a molecular dipole of approximately zero. Examples include hydrocarbons, such as alkanes, alkenes, and alkynes.

In some embodiments, the water-soluble formulation comprises or consists essentially of dietary fatty acid, a non-ionic surfactant, and a phenolic amine. That is, the formulation does not include any water, but optionally may include additional components widely known in the art to be useful in nutraceutical formulations, such as preservatives, taste enhancers, colors, buffers, water, etc. In these formulations, a fat-soluble phenolic amine can be dissolved in the surfactant/fatty acid or oil mixture.

In some embodiments, the water-soluble formulation is a water-solubilized formulation, i.e. it includes a dietary fatty acid, a non-ionic surfactant, a water-soluble phenolic amine, and water (e.g. a water-containing liquid) but does not include organic solvents (e.g. ethanol). The surfactant/fatty acid/water soluble phenolic amine, water complex can self assemble into micelles, once a critical concentration is reached. These micelles are invisible to the naked eye, so that, in some embodiments, the water-solubilized formulation is a transparent water-soluble formulation.

Turning now to the methods described herein, it is noted that when discussing the formulation or the methods of the present disclosure, each of these discussions can be considered applicable to other embodiments, whether or not they are explicitly discussed in the context of that example. Thus, for example, in discussing details about the formulation per se, or the method, such discussion also refers to the other embodiment, and vice versa.

Thus, methods of producing a stable, water-soluble fatty acid formulation with better shelf life are described herein. Simply warming and mixing the dietary fatty acids with a non-ionic surfactant such as glycerol-polyethylene glycol oxystearate will not result in a stable clear water soluble solution that will have a reasonable peroxide value, and good sensory characteristics, such as virtually no smell or taste. This is more typically very true for fish oils, where oxidation will result in a fishy smell and taste, and high PV values.

In addition, without taking care regarding the preparation of the formulations herein, a semi-solid gel-like, cloudy or milky, high viscosity solution is obtained, which can be undesirable. For example, this waxy, cloudy, high viscosity gel is not suitable for forming clear solutions in water or beverages. Rather, it becomes a solidified milky white mass. In contrast, by slowly titrating or adding the dietary fatty acid and warm non-ionic surfactant to warm water, a clear solution can be obtained. The rate at which the dietary fatty acid/surfactant is added to the warm water and the temperature each can each be factors in this process. In one example, the surfactant and water are within the temperature range of 80° F. to 200° F., and often from 80° F. to 120° F. If the resulting dietary fatty acid/surfactant gel mixture is then added to the water too fast, a solid gel-like mass can result. In a particular embodiment, the dietary fatty acid gel is added to water at a rate of from about 0.05 mL/sec to about 25.0 mL/sec. In another particular embodiment, the temperature of the non-ionic surfactant does not exceed 200° F., and is more typically maintained at a temperature of 90° F. to 120° F. The non-ionic surfactant can be stirred thoroughly to remove bubbles (oxygen), and until clear. In a particular embodiment, once the dietary fatty acid has been added to the surfactant, it is stirred for at least 10 minutes, or more, and often for about 1 hour. In a more particular embodiment, the water to which it is to be added is heated to about 100° F. to 150° F. as well, and maintained at about 100° F. while slowly adding the dietary fatty acid gel mixture.

In another aspect, the present disclosure provides for a more stable formulation of a liquid concentrate or beverage comprising dietary fatty acids, with a low peroxide value, better shelf life characteristics, and enhanced consumer acceptance. For example, a beverage made from fish oil omega-3 fatty acids without a fishy odor or taste, or objectionable sensory qualities can be prepared. In addition, stable formulations of dietary fatty acids and oils in liquid concentrates or beverages that do not need to be kept frozen to prevent oxidation are also possible as described herein.

In another aspect, the present disclosure provides a method for enhancing the stability of a dietary fatty acid. The method includes combining a dietary fatty acid and a non-ionic surfactant to form a surfactant-dietary fatty acid mixture, then combining this mixture with a water-soluble phenolic amine such as pyridoxamine. The surfactant-dietary fatty acid-pyridoxamine-water mixture has better shelf life and reduced oxidation during processing and storage (aging).

In another aspect, the present disclosure provides a method of dissolving dietary fatty acid in water. The method includes combining dietary fatty acid with a non-ionic surfactant to form a surfactant-dietary fatty acid mixture. The surfactant-dietary fatty acid mixture is slowly combined with water containing a water-soluble phenolic amine such as pyridoxamine, thereby dissolving the dietary fatty acid in pyridoxamine/water. The surfactant dietary fatty acid mixture is added in this embodiment at a rate not to exceed 5 mL per second to a volume of water of 100 mL, or not more than 5% of the volume of water per second of the volume of water it is being added to. The rate of addition can depend to some degree on the volume of water. The water is to be stirred continuously while the addition of the dietary fatty acid gel is being slowly added. The solution may be heated to increase solubility. The heating temperature is typically selected to avoid chemical breakdown of the dietary fatty acid and/or non-ionic surfactant. The temperature of the dietary fatty acid gel (dietary fatty acid/non-ionic surfactant) should not exceed 200° F., and the water temperature should also not exceed 200° F. Ideally, the temperature of both should be maintained at between 90° F. and 120° F. In some embodiments, the resulting solution is a water-soluble formulation or transparent water soluble formulation as described herein. For example, the resulting solution may be a water soluble formulation that is a crystal clear solution (or clear aqueous solution), with no particles visible to the naked eye.

Also provided are methods of treating a subject using an effective amount of a water soluble formulation such as those described herein. In some embodiments, the subject is a mammalian subject, such as a human or domestic animal.

An effective amount of the water soluble formulation of the present disclosure is an amount sufficient to achieve the intended purpose of a method of the present disclosure, such as treating a particular disease state in a subject (e.g. a human subject).

In accordance with some examples, formulations described herein can be prepared that do not exhibit a peroxide value exceeding about 2.0 meq/Kg during one week of storage under refrigerated conditions, and in some embodiments, it can be less than 1.0 meq/Kg during a week of storage. Likewise, the formulation does not exhibit a peroxide value exceeding about 5.0 meq/Kg during 30 days of sealed storage under ambient conditions. In other words, the formulations are very stable compared to these oils when not prepared as described herein.

In other embodiments, it is noted that the formulation can be prepared as a concentrate, or as a ready to use formulation.

Regarding dosage forms, the amount of dietary fatty acid present in a given formulation should be adequate to treat a disease or provide a health benefit to a subject, which is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age, the desired health benefit, and the like. In calculating the dosage regimen for a patient, the mode of administration also can also be taken into consideration.

The dosage regimen also takes into consideration pharmacokinetics parameters well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient and disease or condition treated.

Single or multiple administrations of dietary fatty acid formulations can be administered depending on the dosage and frequency as required and tolerated by the patient. The formulations should provide a sufficient quantity of active agent to effectively treat the disease state. Lower dosages can be used, particularly when the dietary fatty acid is administered to an anatomically secluded site in contrast to administration orally, into the blood stream, into a body cavity, or into a lumen of an organ. Substantially higher dosages can be used in topical administration. Actual methods for preparing parenterally administrable dietary fatty acid formulations will be known or apparent to those skilled in the art and are described in more detail in such publications as Remington's, supra. See also Nieman, In “Receptor Mediated Antisteroid Action,” Agarwal, et al., eds., De Gruyter, N.Y. (1987).

According to an embodiment of the present disclosure, the dietary fatty acid can be present in the water-soluble formulation at a concentration of from about 0.01 wt % to about 35 wt %. In another embodiment the dietary fatty acid can be present in the water-soluble formulation at a concentration from 1 wt % to 35 wt %. In a more specific embodiment, the dietary fatty acid can be present in the water-soluble formulation at a concentration from 5 wt % to 30 wt %, or more specifically froml 0 wt % to 25 wt %, or still more specifically from 20 wt % to 25 wt %. The dietary fatty acid may also be present (e.g. in a beverage formulation) at a concentration from 0.5 to 1,000 mg per 8 fluid oz. beverage, or around 1-35 mg per mL in a liquid concentrate. In other embodiments, the dietary fatty acid can be present at a concentration from 0.01 mg/mL to 50 mg/mL. In an aspect of the embodiments herein, there can be a maximum concentration for achieving a crystal clear solution. Concentrations of dietary fatty acid above 30 wt % (30 mg/mL) using glycerol-polyethylene glycol oxystearate (i.e. macrogoglycerol hydroxystearate) for example, as the surfactant, will no longer result in a crystal-clear solution in water, though other surfactants have other concentrations where this occurs. Therefore, for dietary fatty acids, the concentration range can be from 0.1 wt % to 25 wt % in the surfactant, or 0.01 mg/mL to 250 mg/mL, with a more specific concentration of around 50 mg/mL. This represents a ratio of dietary fatty acid to surfactant of about 1:4. In some concentrated formulations (e.g. a soft gel capsule formulation), dietary fatty acid may be present at about 1 to 50 mg/mL, or around 20 mg/mL, or at least 1 mg/mL. These numerical values and concentrations are merely exemplary and should not be considered limiting.

In other embodiments, the dietary fatty acid can be present in a water-soluble beverage formulation at from about 0.1 mg to about 1 g. In another embodiment, the dietary fatty acid can be present in the water-soluble formulation in an amount from about 0.1 mg to about 2 g. In a more specific embodiment, the dietary fatty acid can be present at from about 0.5 mg to about 1 g, or even more specifically from about 1 mg to about 500 mg, or still more specifically from about 1 mg to about 50 mg, or still more specifically from about 1 mg to about 5 mg of dietary fatty acid is present in the water-soluble beverage formulation. The phenolic amine can be present in an amount of from about 5 mg to about 500 mg in a solution of 500 mL of liquid. For example, where 250 mg of pyridoxamine is dissolved in 250 mL water, this mixture can be added to a mixture of surfactant and nutritional fatty acids totaling 125 mL (25 mL of fatty acids in 100 mL of surfactant). The total volume of the water-soluble concentrate is then about 375 mL, so the level of the phenolic amine would be about 6.6 mg/mL, or 0.66%. The phenolic amine may be present at a level of from about 0.01 wt % to about 10 wt %. Likewise, if the phenolic amine can be pyridoxamine or an analogue of pyridoxamine, and the concentration may be from about 0.01 wt % to about 0.1 wt %, or alternatively from about 0.5 wt % to about 1 wt %, or still alternatively from about 1 wt % to 5 wt % in solution of water-soluble concentrate.

In some embodiments, the water-soluble formulation is in the form of a pharmaceutical composition. The pharmaceutical composition may include dietary fatty acid, a non-ionic surfactant, a phenolic amine such as pyridoxamine, and a pharmaceutically acceptable excipient. After a pharmaceutical composition including dietary fatty acid of the present disclosure has been formulated in an acceptable carrier, it can be placed in an appropriate container and labeled for treatment of an indicated condition. For administration of dietary fatty acid, such labeling would include, e.g., instructions concerning the amount, frequency, and method of administration.

Any appropriate dosage form is useful for administration of the water-soluble formulation of the present disclosure, such as oral, parenteral and topical dosage forms. Oral preparations include tablets, pills, powder, dragees, capsules (e.g. soft-gel capsules), liquids, lozenges, gels, syrups, slurries, beverages, suspensions, etc., suitable for ingestion by the patient. Examples of liquid formulations are drops, sprays, aerosols, emulsions, lotions, suspensions, drinking solutions, gargles, and inhalants. The formulations of the present disclosure can also be administered by injection, that is, intravenously, intramuscularly, intracutaneously, subcutaneously, intraduodenally, or intraperitoneally. Also, the formulations described herein can be administered by inhalation, for example, intranasally. Additionally, the formulations of the present disclosure can be administered transdermally. The formulations can also be administered by intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995). Thus, the formulations described herein may be adapted for oral administration.

For preparing pharmaceutical compositions from the formulations of the present disclosure, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton Pa. (“Remington's”).

Suitable carriers include magnesium carbonate, magnesium stearate, talc, sugar, lactose, pectin, dextrin, starch (from corn, wheat, rice, potato, or other plants), gelatin, tragacanth, a low melting wax, cocoa butter, sucrose, mannitol, sorbitol, cellulose (such as methyl cellulose, hydroxypropylmethyl-cellulose, or sodium carboxymethylcellulose), and gums (including arabic and tragacanth), as well as proteins such as gelatin and collagen. If desired, disintegrating or co-solubilizing agents may be added, such as the cross-linked polyvinyl pyrrolidone, agar, alginic acid, or a salt thereof, such as sodium alginate. In powders, the carrier is a finely divided solid, which is in a mixture with the finely divided active component. In tablets, the active component is mixed with the carrier having the necessary binding properties in suitable proportions and compacted in the shape and size desired.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the disclosure can also be used orally using, for example, push-fit capsules made of gelatin, as well as soft, sealed capsules made of gelatin and a coating such as glycerol or sorbitol. Push-fit capsules can contain dietary fatty acid mixed with a filler or binders such as lactose or starches, lubricants such as talc or magnesium stearate, and, optionally, stabilizers. In soft capsules, dietary fatty acid may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, beverages, and emulsions, for example, water or water/propylene glycol solutions. For parenteral injection, liquid preparations can be formulated in solution in aqueous polyethylene glycol solution.

Aqueous solutions and beverages suitable for oral use can be prepared by and adding suitable colorants, flavors, stabilizers, and thickening agents as desired. Aqueous suspensions suitable for oral use can be made by dispersing the active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous compositions can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The formulations of the disclosure can be delivered transdermally by a topical route, formulated as applicator sticks, solutions, suspensions, emulsions, gels, creams, ointments, pastes, jellies, paints, powders, and aerosols.

The formulations can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

The formulations of the disclosure can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms.

In another embodiment, the formulations of the disclosure can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the dietary fatty acid, dietary fatty acid metabolite, phenolic amine, pyridoxamine, or salt thereof into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The formulations may be administered as a unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted according to the particular application and the potency of the active component. The composition can, if desired, also contain other compatible therapeutic agents.

Subject non-ionic surfactants may be assayed for their ability to solubilize dietary fatty acid using any appropriate method. Typically, a non-ionic surfactant is warmed and contacted with the dietary fatty acid and mixed mechanically and/or automatically using a shaker, vortex, or sonicator device. Water may be optionally added, for example, where the dietary fatty acid and/or surfactant are in powder form. The solution is heated to increase solubility. The heating temperature is selected to avoid chemical breakdown of the dietary fatty acid or non-ionic surfactant. In a particular example, the surfactant or dietary fatty acid is not heated above 200° F., and more typically, not more than 150° F.

The resulting solution may be visually inspected for colloidal particles to determine the degree of solubility of the dietary fatty acid. Alternatively, the solution may be filtered and analyzed to determine the degree of solubility. For example, a spectrophotometer may be used to determine the concentration of dietary fatty acid present in the filtered solution. Typically, the test solution is compared to a positive control containing a series of known quantities of pre-filtered dietary fatty acid solutions to obtain a standard concentration versus UV/vis absorbance curve. Alternatively, high performance liquid chromatography may be used to determine the amount of dietary fatty acid in solution. Micelles in a size range of from 10 to 100 nm can be measured by light scattering experiments. Typical sizes are from 10 to 50 nm for fatty acid self assembled micelles of the present disclosure.

High throughput solubility assay methods are well known in the art and can be applied to the formulations of the present disclosure. Typically, these methods would involve automated dispensing and mixing of solutions with varying amounts of non-ionic surfactants, dietary fatty acid, and optionally other co-solvents. The resulting solutions may then be analyzed to determine the degree of solubility using any appropriate method as discussed above.

The Millipore MultiScreen Solubility filter plate® with modified track-etched polycarbonate, 0.4 μm membrane is a single-use, 96-well product assembly that includes a filter plate and a cover can be used. The device is intended for processing aqueous solubility samples in the 100-300 μL volume range. The vacuum filtration design is compatible with standard, microtiter plate vacuum manifolds. The plate is also designed to fit with a standard, 96-well microtiter receiver plate for use in filtrate collection. The MultiScreen Solubility filter plate® has been developed and QC tested for consistent filtration flow-time (using standard vacuum), low aqueous extractable compounds, high sample filtrate recovery, and its ability to incubate samples as required to perform solubility assays. The low-binding membrane has been specifically developed for high recovery of dissolved organic compounds in aqueous media.

The aqueous solubility assay allows for the determination of dietary fatty acid solubility by mixing, incubating and filtering a solution in the MultiScreen Solubility filter plate. After the filtrate is transferred into a 96-well collection plate using vacuum filtration, it can be analyzed by Ultraviolet-visible (UV/Vis) spectroscopy to determine solubility. Additionally, LC/MS or HPLC can be used to determine compound solubility, especially for compounds with low UV/Vis absorbance and/or compounds with lower purity. For quantification of aqueous solubility, a standard calibration curve may be determined and analyzed for each compound prior to determining aqueous solubility.

Test solutions may be prepared by adding an aliquot of concentrated drug or compound. In one example, the solutions are mixed in a covered 96-well MultiScreen Solubility filter plate for 1.5 hours at room temperature. The solutions are then vacuum filtered into a 96-well, polypropylene, V-bottomed collection plate to remove any insoluble precipitates. Upon complete filtration, 160 μL/well are transferred from the collection plate to a 96-well UV analysis plate and diluted with 40 μL/well of acetonitrile. The UV/Vis analysis plate is scanned from 260-500 nm with a UV/Vis microplate spectrometer to determine the absorbance profile of the test compound.

Thus, one skilled in the art may assay a wide variety of non-ionic surfactants to determine their ability of solubilize dietary fatty acid compounds.

The terms and expressions which have been employed herein are used as terms of description and not of limitation, and there is no intention in the use of such terms and expressions of excluding equivalents of the features shown and described, or portions thereof, it being recognized that various modifications are possible within the scope of the disclosure claimed. Moreover, any one or more features of any embodiment of the disclosure may be combined with any one or more other features of any other embodiment of the disclosure, without departing from the scope of the disclosure. For example, the features of the formulations are equally applicable to the methods of treating disease or providing other health benefits. All publications, patents, and patent applications cited herein are hereby incorporated by reference in their entirety for all purposes.

### EXAMPLES

The examples below are meant to illustrate certain embodiments of the disclosure, and are not intended to limit the scope herein.

### Example 1

Water-soluble compositions of omega-3 fatty acids were formulated containing the non-ionic surfactant macrogolglycerol hydroxystearate (Glycerol-Polyethylene glycol oxystearate). First, the non-ionic surfactant was heated to about 100° F. and stirred until clear and virtually no bubbles are apparent. A deodorized omega-3 fatty acid fish oil, containing 30 wt % total omega-3 fatty acids at room temperature is very slowly added into the warm macrogolglycerol hydroxystearate until a clear slightly viscous solution was formed containing dissolved omega-3 fatty acids (hereinafter referred to as “omega-3 gel formulation”). The omega-3 gel formulation consisted of macrogolglycerol hydroxystearate (100 mL) and 25 mL (25 grams) of omega-3 fatty acids, representing a concentration of 20 wt % or 200 mg/mL for the omega-3 fatty acids in the non-ionic surfactant. In another vessel, 500 mg of pyridoxamine dihydrochloride is added to 250 mL of warm water (90° F. to 100° F.) until dissolved. The omega-3 gel formulation was slowly titrated, at a rate of about 1 mL per second to the 250 mL of warm water/pyridoxamine solution that was maintained as a mixing vortex with a stirrer at 100 RPM, and maintained at a temperature of about 100° F. until a crystal clear solution was formed. The water was continuously stirred during the addition phase and after until a clear liquid was formed. This solution contained self-assembled micelles containing fatty-acids, surfactant, pyridoxamine, and water.

In further detail, the aqueous omega-3 fatty acid liquid formulation was prepared by maintaining the gel formulation at a temperature of about 100° F. and titrating or adding drop by drop the gel mixture to warm water containing the pyridoxamine salt, to form a “clear aqueous solution” of stabilized omega-3 fatty acids. Upon tasting, the aqueous omega-3 fatty acid formulation did not have undesirable flavor. The aqueous omega-3 fatty acid formulation consisted of water (250 mL), macrogolglycerol hydroxystearate 40 (100 mL), and 30% omega-3 fatty acid fish oil (25 grams), a concentration of omega-3 fatty acids in the aqueous dietary fatty acid formulation of 6.6 wt % or 66 mg/mL (water containing beverage). The aqueous omega-3 fatty acid formulation was analyzed by HPLC to verify content of total fatty acids.

The solution of the present example was tested for peroxide value (PV) according the previously described protocol, and found to have a PV value of less than 0.1 meq/Kg. A sample of the same omega-3 fatty acids used in this example kept refrigerated after defrosting for 1 week had a PV value of 0.2 meq/Kg right after defrosting, and a PV value of 2.5 meq/Kg within 2 weeks. Another sample kept at room temperature, in a sealed, amber glass container, had a PV value of 4.5 meq/Kg after 30 days. Thus, more broadly speaking, the formulation does not exhibit a peroxide value exceeding about 2.0 meq/Kg during one week refrigerated storage. Likewise, the formulation does not exhibit a peroxide value exceeding about 5.0 meq/Kg during 30 days of sealed storage under ambient conditions.

### Example 2

About 5 grams of DHA (docosahexaenoic acid) oil from algae was dissolved in 50 mL of warm Polyethylene Glycol 660 Hydroxystearate by mixing until a clear gel was formed. In another vessel, 250 mg of pyridoxamine dihydrochloride was dissolved in 250 mL of warm water until dissolved. The DHA/surfactant gel was then very slowly added to 250 mL of warm water containing pyridoxamine while mixing with a paddle suspended and rotating at 50 RPM and by slowly adding as a drizzle, or drop-by-drop using a titration apparatus. The DHA/surfactant gel is added very slowly to the mixing water/pyridoxaminer to avoid solidification of the liquid into a solid gel, or cloudy white mass, e.g., at the rate of 1 mL every 10 seconds or more while stirring continues. A clear solution was formed with no visible particles or micelles. This stabilized, water soluble fatty acid solution was tested and found to have a PV value of 0.4 meq/Kg.

What is claimed is:

1. A water-soluble formulation, comprising:
a dietary fatty acid;
a non-ionic surfactant; and
a phenolic amine.

2. The formulation of claim 1, further comprising water.

3. The formulation of claim 1, wherein the dietary fatty acid includes multiple dietary fatty acids.

4. The formulation of claim 1, wherein the dietary fatty acid includes an omega-3 fatty acid.

5. The formulation of claim 1, wherein the omega-3 fatty acid includes eicosapentaenoic acid (EPA), docosahexaenoic acid (DHA), or combination thereof.

6. The formulation of claim 1, wherein the formulation is a non-alcoholic formulation.

7. The formulation of claim 1, wherein the formulation is a non-aprotic solvated formulation.

8. The formulation of claim 1, wherein the dietary fatty acid is present at a total concentration of at least about 0.01 mg/mL.

9. The formulation of claim 1, wherein the dietary fatty acid is present at a total concentration of at least about 1 mg/mL.

10. The formulation of claim 1, wherein the dietary fatty acid is present at a total concentration of at least about 0.01% by weight.

11. The formulation of claim 1, wherein the dietary fatty acid is present at a total concentration of at least about 25% by weight of the formulation.

12. The formulation of claim 1, comprising from about 1 mg to about 250 mg of dietary fatty acid.

13. The formulation of claim 1, comprising at least about 10 mg of dietary fatty acid.

14. The formulation of claim 1, wherein the non-ionic surfactant is selected from the group consisting of non-ionic water soluble mono- and di-fatty acid esters of polyethylene glycol; non-ionic water soluble sorbitan fatty acid esters; polyglycolyzed glycerides; non-ionic water soluble triblock copolymers;
and combinations and derivatives thereof.

15. The formulation of claim 1, wherein the non-ionic surfactant is a non-ionic water-soluble mono-, di-, or tri-glyceride.

16. The formulation of claim 1, wherein the non-ionic surfactant is glycerol-polyethylene glycol oxystearate.

17. The formulation of claim 1, wherein the non-ionic surfactant is macrogolglycerol ricinoleate or macrogolglycerol hydroxystearate.

18. The formulation of claim 1, wherein the non-ionic surfactant is polyethylene glycol 660 hydroxystearate.

19. The formulation of claim 1, wherein the non-ionic surfactant is ethoxylated castor oil.

20. The formulation of claim 1, wherein the phenolic amine is pyridoxamine, a pyridoxamine analogue, or a salt thereof.

21. The formulation of claim 20, wherein the pyridoxamine is a pyridoxamine dihydrochloride salt.

22. The formulation of claim 1, wherein the formulation is an oral formulation.

23. The formulation of claim 22, wherein the oral formulation is a soft gel capsule.

24. The formulation of claim 22, wherein the oral formulation is a beverage.

25. The formulation of claim 1, wherein the formulation is a spray formulation.

26. The formulation of claim 1, wherein the formulation is a topical formulation.

27. The formulation of claim 1, wherein the dietary fatty acid is derived from fish, algae, or vegetable sources.

28. The formulation of claim 1, in which the formulation does not exhibit a peroxide value exceeding about 2.0 meq/Kg during one week refrigerated storage.

29. The formulation of claim 1, in which the formulation does not exhibit a peroxide value exceeding about 5.0 meq/Kg during 30 days of sealed storage under ambient conditions.

30. The formulation of claim 1, in the form of a concentrate.

31. The formulation of claim 1, in the form of a clear aqueous solution.

32. A method of stabilizing dietary fatty acids in water, comprising:
warming a non-ionic surfactant;
combining a dietary fatty acid with the non-ionic surfactant to form a surfactant-dietary fatty acid mixture; and
combining the surfactant-dietary fatty acid mixture with water containing a phenolic amine to form a stabilized, clear, water-soluble, self-assembled fatty acid solution.

33. The method of claim 32, wherein the step of warming is to a temperature from about 80° F. to about 200° F.

34. The method of claim 32, wherein the step of combining the surfactant-dietary fatty acid mixture with water is at a rate from about 0.05 mL/sec to about 25.0 mL/sec.

35. The method of claim 32, wherein the fatty acid solution is a concentrate.

36. The method of claim 32, wherein the non-ionic surfactant is a glycerol-polyethylene glycol oxystearate, ethoxylated castor oil, or polyethylene glycol 660 hydroxysterate, and the phenolic amine is a pyridoxamine salt.

37. A method for enhancing the stability of a dietary fatty acid, comprising:
combining a dietary fatty acid and a non-ionic surfactant to form a surfactant-dietary fatty acid mixture; and
combining the surfactant-dietary fatty acid mixture with a water soluble phenolic amine dissolved in water, resulting in a composition having enhanced stability of the dietary fatty acid compared to the stability of the dietary fatty acid in water alone.

38. A method of making a stable, water-soluble pharmaceutical gel composition of a dietary fatty acid, comprising:
heating a water-soluble non-ionic surfactant in a container to a temperature of about 80° F. to about 200° F. while mixing the non-ionic surfactant until a clear non-ionic surfactant is formed;
adding a dietary fatty acid to the clear non-ionic surfactant to form a dietary fatty acid and non-ionic surfactant combination;
stirring dietary fatty acid and non-ionic surfactant combination until thoroughly mixed so as to constitute from 0.1 wt % to 25 wt % dietary fatty acid and from 70 wt % to 99.9 wt % non-ionic surfactant, wherein the dietary fatty acid is sufficiently dispersed or dissolved in the surfactant so that a gel composition containing no visible micelles or particles of dietary fatty acid is formed;
dissolving a pyridoxamine salt in water in a second vessel; and
adding the gel composition to warm water containing the dissolved pyridoxamine salt at a rate not to exceed 5% of the volume of water per second while continuously stirring the water until a clear solution is formed.

39. The method of claim 38, wherein the non-ionic surfactant is a glycerol-polyethylene glycol oxystearate, ethoxylated castor oil, or polyethylene glycol 660 hydroxysterate, and the phenolic amine is a pyridoxamine salt.

40. The method of claim 38, wherein the pyridoxamine salt is pyridoxamine dihydrochloride.

41. A method of enhancing the stability of a dietary fatty acid in a subject, comprising:
combining a dietary fatty acid and a non-ionic surfactant with a phenolic amine and water to form a mixture of non-ionic surfactant, dietary fatty acid, phenolic amine, and water; and
administering the mixture to a subject, thereby enhancing the stability of the dietary fatty acid or dietary fatty acid metabolite.

42. The method of claim 41, wherein the dietary fatty acid is an omega-3 fatty acid.

43. The method of claim 42, wherein the omega-3 fatty acid is EPA or DHA.

44. A method of administering dietary fatty acid, comprising:
administering a stable, water soluble dietary fatty acid formulation to a subject, the formulation comprising a dietary fatty acid, a non-ionic surfactant, and a phenolic amine.

45. The method of claim 44, wherein the formulation further comprises water.

46. The method of claim 44, wherein the formulation is an oral formulation.

47. The method of claim 46, wherein the oral formulation is a soft gel capsule.

48. The method of claim 46, wherein the oral formulation is a beverage.

49. The method of claim 44, wherein the formulation is a spray formulation.

50. The method of claim 44, wherein the formulation is a topical formulation.
